(19) United States
(12) Plant Patent Application Publication
(10) Pub. No.: US 2003/0150040 P1
(43) Pub. Date: Aug. 7, 2003

(54) Bermuda grass named ' Macho World Feeder '

(76) Inventor: David C. Davidson JR., Poteet, TX (US)

Correspondence Address: JONDLE & ASSOCIATES P.C., 9085 EAST MINERAL CIRCLE, SUITE 200, CENTENNIAL, CO, 80112, US

(21) Appl. No.: 10/062,726

(22) Filed: Feb. 5, 2002

(51) Int. Cl. A01H 5/00

(52) U.S. Cl. PLT/389

(57) ABSTRACT

This invention relates to a new and distinct Bermuda grass (Cynodon dactylon) designated ‘Macho World Feeder’ which is outstanding because of its 1) aggressive growth; 2) its excellent feed values; 3) its salt tolerance; and 4) its drought resistance.

### GENUS AND SPECIES

Bermuda grass (Cynodon dactylon (L.))

### BACKGROUND

‘Macho World Feeder’ is a new and distinct cultivar of Bermuda grass which is developed for forage grass and turfgrass. An important characteristic of a forage grass is that it is good tasting to animals and nutritious by providing most or all the animals dietary needs.

### SUMMARY OF THE INVENTION

This invention relates to a new and distinct Bermuda grass (Cynodon dactylon) designated ‘Macho World Feeder’ which is outstanding because of its 1) aggressive growth; 2) its excellent feed values; 3) its salt tolerance; and 4) its drought resistance. The claimed Bermuda grass ‘Macho World Feeder’ was primarily selected for these characteristics.

The present invention is a mutant that was discovered in August, 2000 after the inventor noticed an area of grass which was distinctly taller and larger-leafed and stemmed than the surrounding grass which was ‘World Feeder’ Bermuda Grass (U.S. Plant Pat. No. 7,081). The grass was selected, separated and propagated during September and October.

### DEFINITIONS

In the description and tables which follow, a number of terms are used. In order to provide a clear and consistent understanding of the specification and claims, including the scope to be given such terms, the following definitions are provided:

Total Digestible Nutrients (TDN)—As used herein, the term “total digestible nutrients” is a measurement of the energy value of a feed or forage grass.

Acid Detergent Fiber (ADF)—As used herein, the term “acid detergent fiber” is a measurement of lignin and cellulose fiber content of a feed or forage which helps determine its digestibility. The desired range is between 28%-32%.

### ORIGIN AND ASEXUAL REPRODUCTION

Asexual reproduction of this turfgrass by vegetative propagation was directed by me, such reproduction establishing that the plant is stable and in fact maintain the characteristics described, in successive generations.

### BRIEF DESCRIPTION OF THE DRAWINGS

The accompanying photographs show as nearly true as it is reasonably possible to make the same, in color illustrations of this character, the new variety. All comparisons in these photographs were from samples that were planted at the same time and with identical environmental conditions.

FIG. 1 shows ‘Macho World Feeder’ Bermuda grass growing in a field next to the parental cultivar ‘World Feeder’ Bermuda Grass (U.S. Plant Pat. No. 7,081).

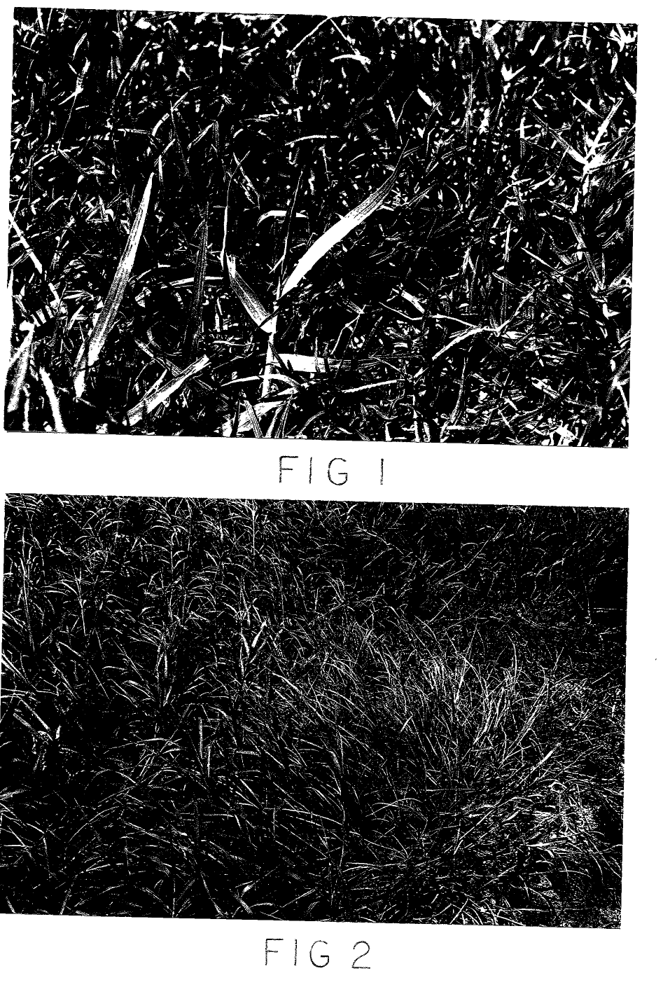

FIG. 2 shows ‘Macho World Feeder’ Bermuda grass growing in a field next to ‘Coastal’ Bermuda grass.

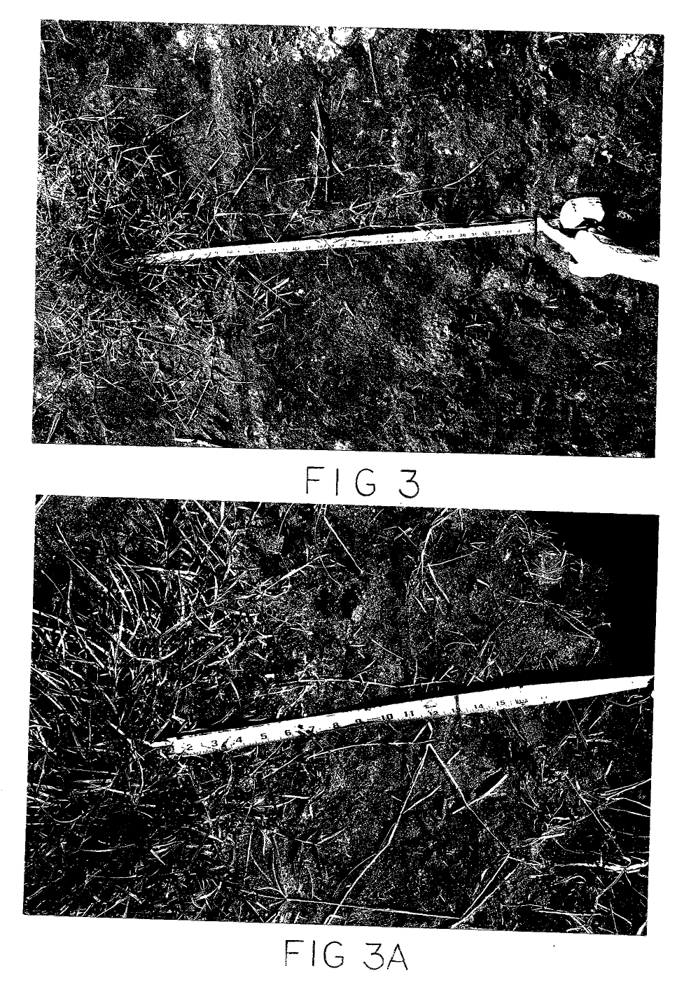

FIG. 3 and FIG. 3A show measurements of runners from ‘Macho World Feeder’ Bermuda grass and parental cultivar ‘World Feeder’ Bermuda Grass respectively.

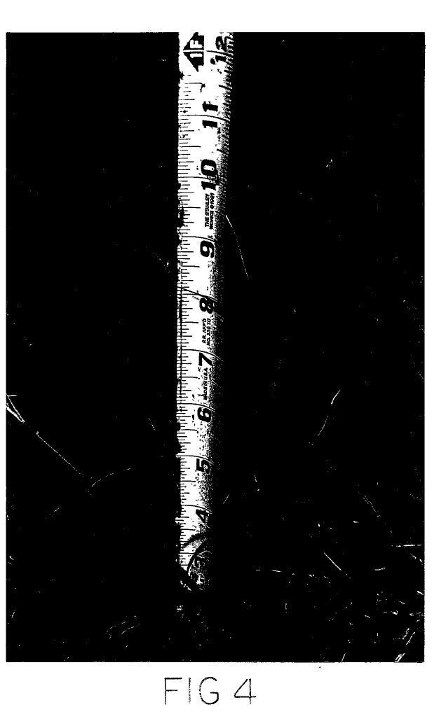

FIG. 4 and FIG. 4A show vertical growth of ‘Macho World Feeder’ and ‘World Feeder’ Bermuda Grass respectively.

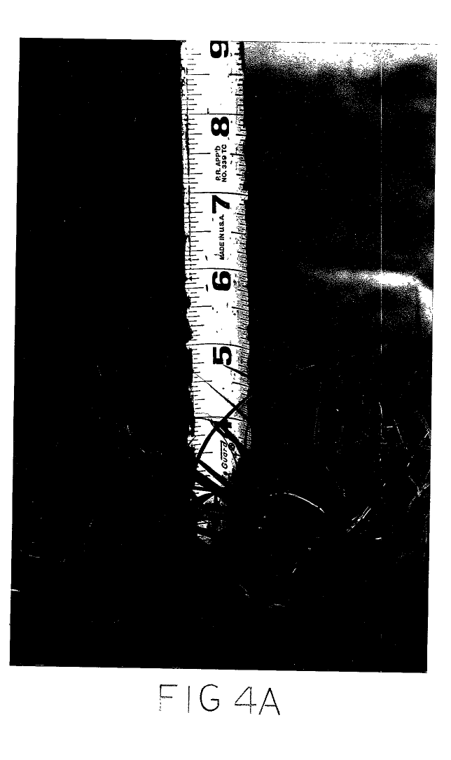

FIG. 5 and FIG. 5A show the blade length of ‘Macho World Feeder’ and ‘World Feeder’ Bermuda Grass respectively.

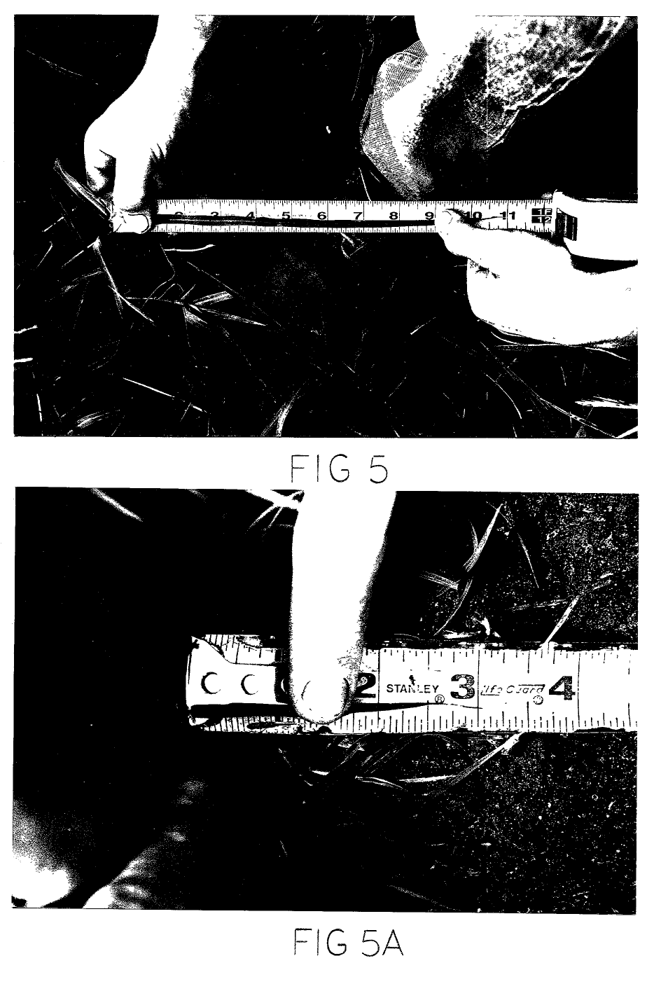

### DETAILED DESCRIPTION OF THE INVENTION

Parentage: ‘World Feeder’ Bermuda Grass (U.S. Plant Pat. No. 7,081)

Propagation: Vegetative cuttings or sod

Growth Habit:

Runners.—Stem distance between nodes: Average 14.8 cm on horizontal runners; width of runner is 2-3 mm; daily growth over 3 day period is 9 cm.

Leaves.—Leaf width averages 4-5 mm; leaf length averages 17 cm with several being 26-30 cm.

Observations: Grass grew approximately 15-16 inches over a 5-day period in the spring.

Cold tolerant.—survived Texas and Oklahoma winters; further testing in Nebraska and Iowa.

Salt tolerant.—‘Macho World Feeder’ has survived 30,000 parts per million salt through two cutting cycles.

Feed Values: At premium harvest age, protein levels of 26.8% have been obtained. A sample taken 10 days later measured 22.4% protein and a sample that was allowed to head out still measured 22% protein. Total digestible nutrients (TDN) measured from 68.6% to 72.6% and acid detergent fiber (ADF) measured from 26.3% to 29.8%. One plot was allowed to grow uncut from early March to July 10. The protein was still 10.8%, ADF 38.7% and TDN 58.4%.

Comparison With Parental Cultivar: ‘Macho World Feeder’ grows approximately 300-400% faster than ‘World Feeder’ Bermuda grass (U.S. Plant Pat. No. 7,081). The leaf blades and runners are much larger than ‘World Feeder’. Additionally, ‘Macho World Feeder’ has a higher protein percent over ‘World Feeder’ Bermuda Grass.

What is claimed is:

1. A new and distinct Bermuda Grass as described and illustrated herein.
